(19) United States
(12) Patent Application Publication
(10) Pub. No.: US 2006/0147486 A1
(43) Pub. Date: Jul. 6, 2006

(54) Biodegradable dual porous scaffold wrapped with semi-permeable membrane and tissue cell culture using thereof

(76) Inventors: Jung-Hyun Kim, Seoul (KR); Hye-Won Lee, Seoul (KR); Sung-Wook Choi, Seoul (KR)

Correspondence Address: SHERIDAN ROSS PC, 1560 BROADWAY, SUITE 1200, DENVER, CO, 80202, US

(21) Appl. No.: 10/542,427

(22) PCT Filed: Jan. 14, 2004
(86) PCT No.: PCT/KR04/00054

§ 371 Date: Mar. 9, 2006

(30) Foreign Application Priority Data
Jan. 14, 2003 (KR) 10-2003-0002314

(51) Int. Cl. A61F 13/00; C12N 5/08

(52) U.S. Cl. 424/422; 435/366; 435/289.1

(57) ABSTRACT

Disclosed is a scaffold including a semi-permeable membrane on an outer surface thereof. The present invention also discloses a method of preparing a scaffold covered with a semi-permeable membrane, including loading one or more scaffolds into a mold with a predetermined form and size; and adding a semi-permeable agent and a cross-linking agent to the mold and cross-linking the semi-permeable agent to form the semi-permeable membrane on the outer surface of each of the scaffolds. The scaffold covered with the semi-permeable membrane selectively introduces nutrients into the scaffold by allowing penetration of only external nutrients into the scaffold and excreting metabolic wastes generated by tissue cells to the outside of the scaffold. In addition, the scaffold has the morphology of a biological tissue of interest by cross-linking the small-sized scaffolds, thereby allowing uniform proliferation of tissue cells throughout the whole scaffold.

### TECHNICAL FIELD

The present invention relates, in general, to a scaffold and a method of preparing biological tissues using the scaffold. More particularly, the present invention relates to regeneration of biological tissues by preparing a porous scaffold by gas foaming of an effervescent salt using a biodegradable polymer, sectioning the scaffold into small pieces, seeding tissue cells onto the scaffold pieces, forming a semi-permeable membrane on an outer surface of each of the scaffold pieces, and cross-linking the semi-permeable membrane-covered scaffold pieces into a predetermined form.

The term “scaffold”, as used herein, refers to a porous biodegradable polymer construct to support cell growth and migration.

### BACKGROUND ART

Typically, bone cartilage is a tissue that is not naturally regenerated once damaged. To repair damaged cartilage tissues, cartilage substitutes such as non-absorbable biological substances have been used. However, the non-absorbable cartilage substitutes used up to date develop various side effects and complications, such as skin necrosis and inflammation. For this reason, cartilage autografts are recognized as the best implant materials.

Recently, efforts were made to reconstruct damaged biological tissues by regenerating a portion of the damaged tissues in laboratories. This approach, defined as “tissue engineering”, has raised tremendous attention.

Tissue engineering involves the development of a novel generation of biocompatible materials capable of specifically interacting with biological tissues to produce functional tissue equivalents. Tissue engineering has a basic concept of collecting a desired tissue from a patient, isolating cells from the tissue specimen, proliferating the tissue cells up to a desired quantity by cell culturing, seeding the proliferated cells onto a biodegradable polymeric scaffold with a porous structure, culturing the cells for a predetermined period in vitro, and transplanting back the cell/polymer construct into the patient.

After the above procedure, the cells in the transplanted scaffold, in most tissues or organs, use oxygen and nutrients gained by diffusion of body fluids until new blood vessels are generated in the scaffold. As blood vessels extend into the scaffold, the cells proliferate and differentiate to form a new tissue and a new organ whereas the scaffold has been dissolved.

The scaffold used for the regeneration of biological tissues, as described above, is made of a material satisfying the major requirements, as follows. The material should sufficiently serve as a template or matrix to allow tissue cells to attach to the surface of the material and form a three-dimensional tissue. Also, the material should act as a barrier that is positioned between the seeded cells and host cells. These requirements mean that the material should be nontoxic and biocompatible, that is, does not cause blood clotting or inflammation after being transplanted.

Further, the material for preparation of the scaffold should have biodegradability to allow for being completely degraded and eventually extinguished in vivo as the transplanted cells sufficiently perform their innate functions and roles as a tissue.

The most widely used biodegradable polymers, satisfying the aforementioned physical requirements, include polyglycolic acid (PGA), polylactic-co-glycolic acid (PLGA), poly-ε-caprolactone (PCL), polyamino acids, polyanhydrides, polyorthoesters and copolymers thereof.

On the other hand, the aforementioned polymers have been researched to fabricate porous scaffolds. Several techniques have been utilized for scaffold fabrication, such as solvent-casting and particulate-leaching including the steps of mixing the polymer dissolved in an appropriate solvent with single crystal salt particles, evaporating the solvent from the polymer/salt composite and immersing the solidified sample in distilled water for leaching of the salt particles (A. G. Mikos et al., Polymer, 35, 1068, 1994); gas foaming based on the use of high-pressure CO₂ gas to foam a polymer (L. D. Harris et al., Journal of Biomedical Materials Research, 42, 396, 1998); fiber extrusion and fabric forming processing based on extrusion of a polymer fiber into a non-woven fabric and then formation of a polymer mesh (K. T. Paige et al., Tissue Engineering, 1, 97, 1995); thermally induced phase separation based on formation of pores by immersing a polymer solution in a non-solvent to exchange a solvent in the polymer solution for the non-solvent (C. Schugens et al., Journal of Biomedical Materials Research, 30, 449, 1996); and emulsion freeze-drying including mixing a polymer solution and water, quenching the resulting emulsion in liquid nitrogen and subsequently freeze-drying the emulsion (K. Whang et al., Polymer, 36, 837, 1995).

However, the conventional fabrication techniques generally result in scaffolds with relatively low porosities, uncontrollable pore size and poorly interconnected, open-pore networks. Also, when tissue cells are seeded onto the scaffold and proliferated thereon, the pores on the surface of the scaffold are often blocked, thereby causing difficulty in preparation of grafts. Thus, the conventional techniques further include the following problems: toxic gas can be generated during the fabrication of the scaffold; slats remain in the scaffold; cells have difficulties in growth into the scaffold; and nutrients are not sufficiently supplied to the cells.

Tissue cells seeded onto the scaffold with an interconnected pore structure, fabricated with the aforementioned biodegradable polymers, grow on the scaffold and form a tissue. Typically, regeneration of a biological tissue is achieved by preparing a scaffold with the morphology of the biological tissue, seeding tissue cells onto the scaffold and allowing the growth of the seeded tissue cells.

However, this method of preparing a graft has disadvantages, as follows. Nutrients and oxygen are not easily transported into the scaffold. Also, the tissue cells do not grow uniformly throughout the scaffold. Even if the scaffold is very thin, the tissue cells have difficulty in growing into the central region of the scaffold.

### DISCLOSURE OF THE INVENTION

To solve the aforementioned problems, the present invention aims to provide a scaffold having a semi-permeable membrane on an outer surface thereof.

In addition, the present invention aims to provide a method of forming a semi-permeable membrane on an outer surface of a scaffold by cross-linking of alginate.

Further, the present invention aims to provide a method of proliferating tissue cells, including sectioning a scaffold into small pieces; seeding tissue cells onto each of the scaffold pieces and loading the scaffold pieces into a mold having a morphology of a tissue to be regenerated; adding a mixture of a semi-permeable agent and a cross-linking agent to the mold loaded with the scaffold pieces and cross-linking the semi-permeable agent surrounding each of the scaffold pieces to form a semi-permeable membrane on an outer surface of each of the scaffold pieces; and introducing nutrients into the resulting scaffold via the semi-permeable membrane.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flow chart showing a method of preparing a scaffold by gas foaming of an effervescent salt;

FIG. 2 is a flow chart showing a method of preparing a scaffold having a semi-permeable membrane on an outer surface thereof according to the present invention;

FIG. 3 is a scanning electron microscopy (SEM) image showing a surface of a scaffold according to the present invention;

FIG. 4 is a SEM image showing an inner surface with porous interconnections of a scaffold according to the present invention;

FIG. 5 shows the actual images and sizes of scaffolds according to the present invention;

FIG. 6 shows SEM images showing chondrocytes that have been proliferated on a scaffold covered with a semi-permeable membrane according to the present invention;

FIG. 7 is a graph showing DNA synthesis of chondrocytes grown on a scaffold covered with a semi-permeable membrane according to the present invention;

FIG. 8 is a graph showing glycosaminoglycan synthesis of chondrocytes grown on a scaffold covered with a semi-permeable membrane according to the present invention; and

FIG. 9 shows results of hematoxylin-eosin staining, masson-trichrome staining and Alcian Blue staining of scaffolds according to present invention, which were implanted into nude mice and then retrieved from the nude mice after four weeks.

### BEST MODE FOR CARRYING OUT THE INVENTION

In one aspect, the present invention is characterized in that a scaffold has a semi-permeable membrane on an outer surface thereof.

In another aspect, the present invention provides a method preparing a scaffold covered with a semi-permeable membrane, including loading one or more scaffolds into a mold with a predetermined form and size; and adding a semi-permeable agent and a cross-linking agent to the mold and cross-linking the semi-permeable agent to form the semi-permeable membrane on an outer surface of each of the scaffolds.

In a further aspect, the present invention provides a method of preparing a biological tissue, including seeding cells obtained from a tissue to be regenerated onto one or more scaffolds; loading the scaffolds seeded with the tissue cells into a molding container with a predetermined form and size; adding a semi-permeable agent and a cross-linking agent to the molding container and forming a semi-permeable membrane on an outer surface of each of the scaffolds loaded in the molding container to interconnect the scaffolds; and introducing nutrients into the scaffolds interconnected with the cross-linking agent, thus proliferating the tissue cells.

The scaffold according to the present invention is a device designed as a cell carrier for tissue engineering or associated applications, and is mostly in sponge form. Preferably, “scaffold” means a biodegradable polymer construct with a porous structure to support migration and proliferation of cells, and is usually called “polymer support” having biodegradability.

In particular, the aforementioned porosity refers to a plurality of pores that indicate spaces found in tabular pore walls and spaces between polymer struts. The pore size ranges from 200 to 350 μm, and preferably, 230 to 270 μm.

In addition, the pores include micropores that distributes on a wall surface of the scaffold and have a pore size of less than 2 μm.

In addition, any scaffold, which is capable of providing a place where tissue cells are grown after being seeded onto that place, can be used in the present invention. Also, if widely used by those skilled in the art, any scaffold may be employed in the present invention.

Examples of fabrication techniques for the scaffold include solvent-casting and particulate-leaching including the steps of mixing the polymer dissolved in an appropriate solvent with single crystal salt particles, evaporating the solvent from the polymer/salt composite and immersing the solidified sample in distilled water for leaching of the salt particles; gas foaming based on the use of high-pressure CO₂ gas to foam a polymer; fiber extrusion and fabric forming processing based on extrusion of a polymer fiber into a non-wover fabric and then formation of a polymer mesh; thermally induced phase separation based on formation of pores by immersing a polymer solution in a non-solvent to exchange a solvent in the polymer solution for the non-solvent; and emulsion freeze-drying including mixing a polymer solution and water, quenching the resulting emulsion in liquid nitrogen and subsequently freeze-drying the emulsion. Gas foaming using an effervescent salt is recommended, whereby a scaffold is fabricated by dissolving an effervescent salt in a polymer.

Hereinafter, the scaffold prepared by gas foaming of an effervescent salt will be described.

The scaffold according to the present invention is made of a biodegradable polymer which is not toxic to the body and has no side effects when applied to the body and is biodegraded by metabolism. Examples of the biodegradable polymer include polyglycolic acid (PGA), polylactic acid (PLA), polylactic-co-glycolic acid (PLGA), poly-ε-caprolactone (PCL), polyamino acids, polyanhydrides, polyorthoesters and copolymers thereof. The preferred biodegradable polymer is PLGA having a molecular weight of 90,000 to 126,000 Da.

The applicable effervescent salt used for the fabrication of the scaffold according to the present invention, which functions to control the pore size in the scaffold, is such as ammonium hydrogen carbonate (NH₄HCO₃), ammonium carbonate ((NH₄)₂CO₃), sodium hydrogen carbonate (NaHCO₃) and sodium carbonate (Na₂CO₃).

Organic solvent used in the present invention is any substance capable of dissolving a biodegradable polymer and yielding a highly viscous polymer solution. The organic solvent is a mixture of one selected from among dimethylsulfoxide (DMSO), acetonitrile (CH₃CN), dimethylformamide (DMF), N-methylpyrrolidone, etc. and one selected from among methylenechloride (CH₂Cl₂), chlroroform (CHCl₃), acetone, acetic acid (CH₃COOH), tetrahydrofuran (THF), ethylacetate, methylethylketone (MEK), dioxane, dioxane/water, etc. The mixture of dimethylsulfoxide and methylenechloride is recommended.

Herein, in case of using the mixture of dimethylsulfoxide and methylenechloride for scaffold fabrication,the size of micropores is formed smaller as the content rate of dimethylsulfoxide gets lower in the mixture. However, when only methylenechloride is used as a solvent, micropores are not generated.

On the other hand, the scaffold according to the present invention, in a state of being covered with a semi-permeable membrane, serves as a substrate for the growth of tissue cells, wherein the semi-permeable membrane refers to a membrane produced by cross-linking of a semi-permeable agent.

This semi-permeable membrane, which is formed on an outer surface of the scaffold seeded with tissue cells, selectively introduces nutrients into the scaffold from the outside of the scaffold, as well as excreting metabolic wastes generated by the tissue cells to the outside of the scaffold. The semi-permeable membrane generated by the cross-linking of a semi-permeable agent is resulted from the reaction between the semi-permeable agent and a cross-linking agent. Any material capable of performing the aforementioned functions of the semi-permeable membrane may be used as the semi-permeable agent, such as alginates, polysaccharides, chitosan, agar powder and gelatin. The most preferred semi-permeable agent is alginate.

The alginate is a salt of alginic acid, such as sodium alginate, and is a linear copolymer composed of β-D-mannuronic acid and α-L-gluronic acid, which exist in various arrangements in the polymer chain. In addition, the alginate possesses biocompatibility. Due to the biocompatibility, the alginate is widely used as a condensing agent, an emulsifier and the like in the food industries, and further used in bioengineering-related applications including polymeric films, ointments and surgical gauze and also applied for immunoprotection of living cells such as gel beads encapsulating the living cells (P. Aebischer et al., Transplantation in humans of encapsulated xenogenein cells without immunosuppresson, a preliminary report, Transplantation, 58, 1275-1277(1994); P. De Vos et al., Improved biocompatibility but limited graft survival after purification of alginate for microencapsulation of pancreatic islets, Diabetologia 40, 262-270(1997); G. Klock et al., Production of purified alginates suitable for use in immoisolated transplantation, Appl. Microbiol. Biotechno. 40, 638-643(1994)).

In addition, the alginate is water-soluble, and cross-linked by a divalent cation solution, for example, a cross-linking agent such as calcium chloride. Herein, the calcium ion is linked where the gluronic acid of the alginate chain is located to form a stable alginate gel.

In particular, the rate of the rapid gelation carried out by the divalent cation solution is affected by the density of the cross-linkage and the concentrations of polymers based on the gel beads. Typically, as concentrations of the semi-permeable agent and the cross-linking agent are increased and the cross-linking reaction is carried out for a longer time, the hardness of the semi-permeable membrane increases. Therefore, according to the present invention the usable rate of the concentration of the semi-permeable agent ranges from 0.5 to 5%, preferably, 1 to 2%, and more preferably, 1.5%, while the concentration of the cross-linking agent ranges from 1 to 5%, preferably, 1 to 2%, and more preferably, 1.1%. Also, the reaction time is 1 to 20 min, and preferably, 5 to 10 min.

On the other hand, the ratio of β-D-mannuronic acid to α-L-gluronic acid affects the biocompatibility and structure of the alginate beads. The alginate beads have low cytotoxicity and reduce reticulocyte destruction when contacting with the blood (P. De Vos, B. De Haan, R. Van Schilfgaarde., Effect of the alginate composition on the biocompatibility of alginate-polylysine microcapsules, Biomater. 18, 273-278(1997); D. Joseph et al., The biomedical engineering handbook, CRC Press, IEEE Press, 1788-1806(1995)).

The cross-linking agent used for the formation of the semi-permeable membrane according to the present invention primarily form the semi-permeable membrane, as well as interconnecting scaffold pieces 1 to 3 mm in size, and preferably, 1.5 to 2.5 mm in size, thereby allowing for the scaffold to have the morphology of a biological tissue to be generated. That is, the cross-linking agent interconnects the scaffold pieces by cross-linking, and thus, gelating a semi-permeable agent on a surface of the scaffold pieces. Calcium chloride, tripolyphosphate and glutaraldehyde can be used for cross-linking agent capable of performing the aforementioned functions. The calcium chloride is preferred the most.

Fabrication of the scaffold having the aforementioned organization according to the present invention and the method of regenerating a biological tissue using the scaffold will be described in detail, below.

First, a method of preparing a scaffold, for example, by gas foaming of an effervescent salt will be described. Then, a method of preparing a scaffold covered with a semi-permeable membrane using the above scaffold fabricated by the gas foaming will be described.

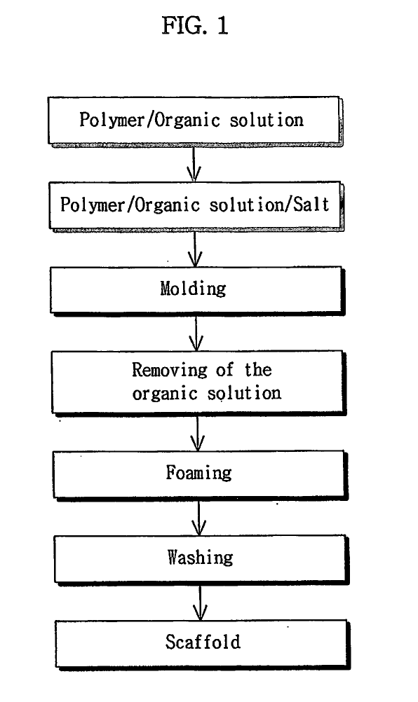

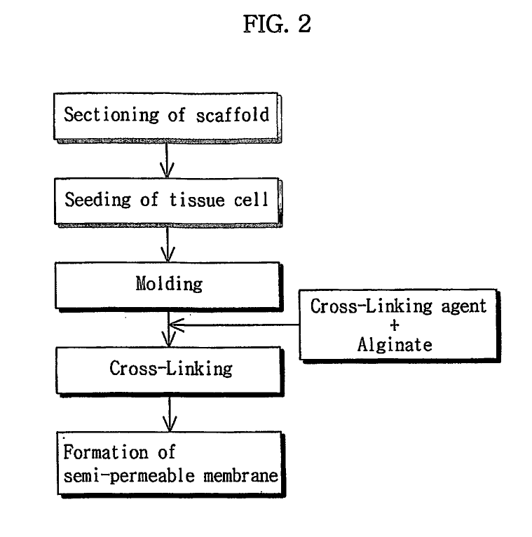

FIG. 1 is a flow chart showing a method of preparing a scaffold by gas foaming of an effervescent salt, while FIG. 2 is a flow chart showing a method of preparing a scaffold having a semi-permeable membrane on an outer surface thereof according to the present invention.

Preparation of a Scaffold By Gas Foaming of an Effervescent Salt

As shown in FIG. 1, a method of preparing a scaffold according to present invention includes:

(i) dissolving a biodegradable polymer in an organic solvent to provide a polymer solution with high viscosity;

(ii) adding an effervescent salt to the polymer solution to provide a polymer gel of copolymer/organic solvent/salt;

(iii) formulating shaping the polymer gel into a mold with a predetermined form and size;

(iv) removing the organic solvent from the copolymer gel made of copolymer/organic solvent/salt;

(v) immersing the copolymer/salt mixture in a solvent for salt leaching and gas foaming; and

(vi) washing the resulting scaffold.

The fabricated scaffold is sectioned into pieces 1 to 3 mm in size, and preferably 1.5 to 2.5 mm in size, for use to regenerate biological tissues. This scaffold sectioning is carried out for regeneration of biological tissues, not in the conventional up-down manner in which a scaffold is originally prepared with a size suitable for the morphology of a biological tissue to be generated, but in a bottom-up manner in which small scaffold pieces are arranged into the desirable morphology of a biological tissue and tissue cells are then proliferated on the resulting scaffold.

On the other hand, the scaffold prepared as described above has a dual porous structure and an improved mechanical property, and thus, allows for the uniform distribution of tissue cells in the scaffold.

In particular, pores and micropores comprising the above dual porous structure are formed by controlling the content of the organic solvent used and using pore-forming solid particles with a certain size.

The dual porous structure of the scaffold according to the present invention is obtained by the following procedure.

First, to generate micropores on the scaffold, dimethylsulfoxide and methylenechloride are mixed in a predetermined ratio in the organic solvent of the above step (i), and the biodegradable polymer is dissolved in the mixture. To generate pores, the resulting polymer solution is supplemented with pore-forming solid particles having a certain size such as ammonium hydrogen carbonate, ammonium carbonate, sodium hydrogen carbonate or sodium carbonate.

Then, after the methylenechloride evaporates, the above mixture is immersed in water, ethanol, methanol or a mixture thereof to leach out the dimethylsulfoxide.

During the dimethylsulfoxide leaching, micropores are generated on the scaffold.

On the other hand, as the content of the dimethylsulfoxide is reduced, the micorpores are formed in smaller size. When only the methylenechloride is used as a solvent, micropores are not formed. In particular, in case of using a solvent composed of 5% dimethylsulfoxide and 95% methylenechloride, micropores of about 10 μm in size are formed. In case of using a solvent of 1% dimethylsulfoxide and 99% methylenechloride, micropores of about 2 μm in size are formed.

In addition, the dimethylsulfoxide may be replaced by a solvent such as acetonitrile (CH₃CN), dimethylformamide (DMF) or N-methylpyrrolidone (NMP).

Preparation of a Scaffold Having a Semi-permeable Membrane on an Outer Surface Thereof

As shown in FIG. 2, a method of preparing a scaffold covered with a semi-permeable membrane using the scaffold prepared by gas foaming of an effervescent salt includes:

(i) seeding cells from a tissue to be regenerated onto one or more scaffold pieces;

(ii) loading the scaffold pieces seeded with the tissue cells into a molding container with a predetermined form and size; and

(iii) adding a semi-permeable agent to the molding container and cross-linking the semi-permeable agent using a cross-linking agent to interconnect the scaffold pieces loaded in the molding container.

Wherein, at step (iii), a mixture of a cross-linking agent and alginate, if desired, may be supplemented with a buffer solution.

Meanwhile, at step (iii), it is ideal to use the semi-permeable agent containing a concentration of 0.5 to 5%, preferably, 1 to 2%, and more preferably, 1.5%, the cross-linking agent containing a concentration of 1 to 5%, preferably, 1 to 2%, and more preferably, 1.1%. The ideal reaction time for cross-linking is 1 to 20 min, and preferably, 5 to 10 min.

With the above scaffold covered with a semi-permeable membrane a biological tissue is regenerated by the following procedure.

To regenerate a biological tissue using the scaffold covered with a semi-permeable membrane according to the present invention, the primarily prepared scaffold is sectioned into pieces with 1 to 3 mm thick, and preferably, 1.5 to 2.5 mm thick, to provide a plurality of scaffold pieces. Then, tissue cells are seeded onto each of the scaffold pieces.

Thereafter, the scaffold pieces seeded with tissue cells are mixed with a semi-permeable agent, and loaded into a mold with the morphology of the tissue to be regenerated to allow for the scaffold pieces to have the tissue morphology.

Subsequently, a cross-linking agent is slowly added to the mold containing the scaffold pieces therein to cross-link the semi-permeable agent, and thus, provide a semi-permeable membrane on the outer surface of the scaffold pieces. A culture medium is added to the scaffold with a semi-permeable membrane on its outer surface, and the tissue cells present in the scaffold are then able to grow.

During the growth of the tissue cells, the semi-permeable membrane excretes metabolic wastes generated by the tissue cells to the outside of the scaffold and selectively penetrates nutrients and oxygen, present outside of the scaffold, into the scaffold.

Meanwhile nutrients and oxygen that have penetrated the semi-permeable membrane are supplied to tissue cells via pores formed on the surface of the scaffold. With the help of nutrients and oxygen, the tissue cells uniformly grow throughout the scaffold, and thus, regenerate a biological tissue.

The scaffold covered with a semi-permeable membrane according to the present invention may be applied for three-dimensional cell cultures in tissue engineering such as tissue or organ regeneration processes. In more detail, the scaffold of the present invention may be utilized as a cell culture structure for cartilage regeneration, a cell culture structure for bone tissue regeneration, a tubular cell culture structure for neovascularization, a tubular cell culture structure for nerve regeneration, a cell culture structure for regeneration of damaged tissues, a cell culture structure for regeneration of organs (heart, lung, liver, etc.) using porous polymer membranes and stem cells, and the like.

The present invention will be explained in more detail with reference to the following examples in conjunction with the accompanying drawings. However, the following examples are provided only to illustrate the present invention, and the present invention is not limited to the examples.

### EXAMPLE 1

### Scaffold Fabrication

A mixture solvent was prepared by mixing 90% of methylchloride (Ducksan Chemical Co. Ltd., Korea) and 10% of dimethylsulfoxide (Sigma, USA). 10% of PLGA, based on the weight of the mixture solvent, which was composed of lactic acid (Sigma, USA) and glycolic acid (Sigma, USA) at a weight ratio of 75:25, molecular weight of 90,000 to 126,000 Da, was dissolved in the mixture solvent.

The resulting solution was mixed with ammonium hydrogen carbonate (Junsei Chemical Co. Ltd., Japan) having a particle size of 150-250 μm at a weight ratio of 9:1. The resulting mixture was poured into a cylinder-type schale in size of 100 mm in diameter (Dongsung science Co. Ltd, Korea).

The methylchloride (Ducksan Chemical Co. Ltd., Korea) contained in the mixture filled into the schale (Dongsung Science Co. Ltd, Korea) was partially evaporated, thus generating a semi-solidified sample.

Thereafter, the semi-solidified sample was immersed in 30% acetic acid in ethanol until foam was not produced.

The resulting sample was then washed with distilled water for three hours and dried in a drier for several days, thus generating a scaffold.

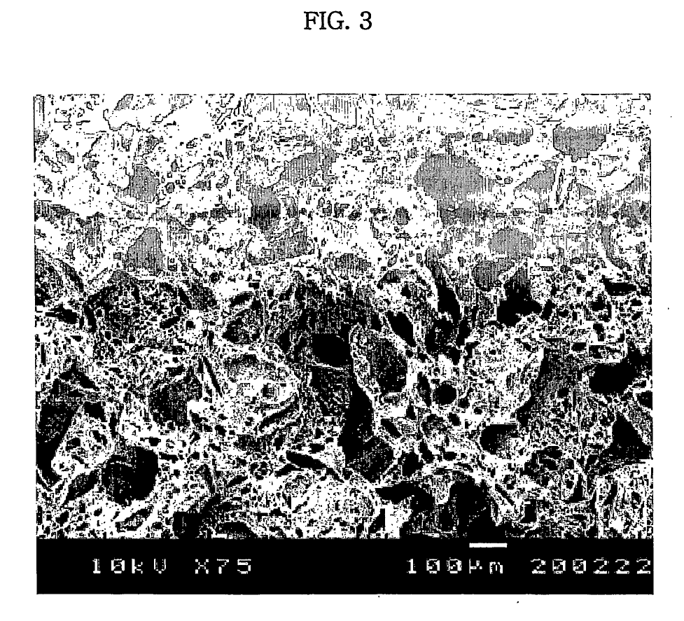

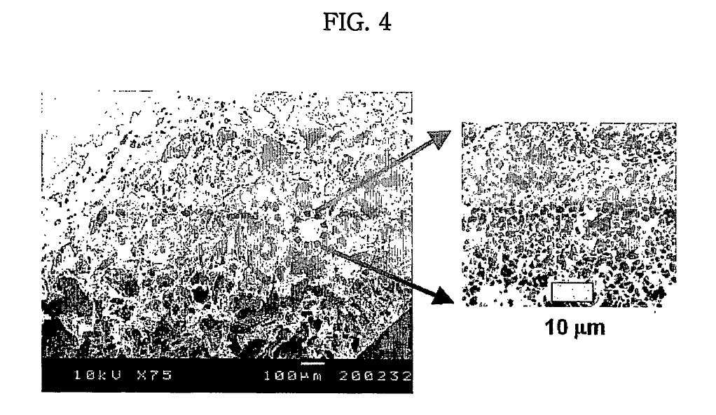

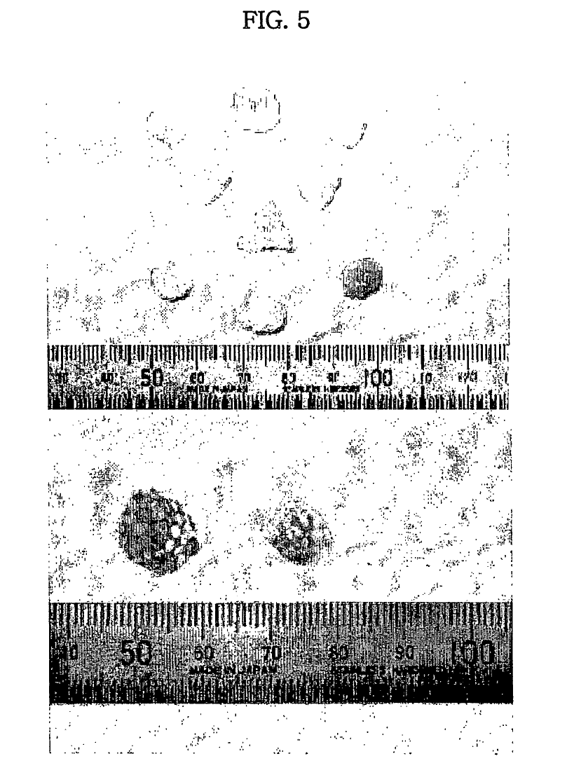

The fabricated scaffold size was 100 mm in diameter and 1 mm thick. The scaffold was observed under scanning electron microscope (SEM, JSM-5410LV, Jeol, Japan), and the resulting images are given in FIGS. 3 and 4. Also, the actual images and sizes of the scaffolds is given in FIG. 5.

FIG. 3 is a scanning electron microscopy (SEM) image showing a surface of a scaffold according to the present invention, while FIG. 4 is a SEM image showing an inner surface with porous interconnections of a scaffold according to the present invention. FIG. 5 shows the actual image and size of scaffolds according to the present invention.

As shown in FIG. 3, the scaffold was found to have a porous structure. As shown in FIG. 4, micropores for cell seeding were formed on an inner surface with porous interconnections of the scaffold, while the micropores were interconnected.

### EXAMPLE 2

### Regeneration of a Biological Tissue

(i) Seeding of Tissue Cells Onto the Scaffold

The scaffold prepared in Example 1 was sectioned into pieces at the size of 2 mm in diameter and 1 mm thick. Chondrocytes isolated from rabbits were seeded onto the scaffold pieces at a density of 1.0×10⁶ cells/ml.

The scaffold pieces were incubated at 37° C. in high humidity containing 5% concentration of CO₂ for about three hours to allow for cross-linking of the scaffold pieces. Then, they were incubated for three days in DMEM (JBI, Korea) containing 4 ml of an antibiotic/antifungal solution and FBS (Sigma, USA).

(ii) Formation of a Semi-permeable Membrane on an Outer Surface of the Scaffold and Proliferation of the Tissue Cells.

A 3% sodium alginic acid solution (Sigma, USA) was mixed with an equal volume of DMEM (JBI, Korea) containing 4 ml of an antibiotic/antifungal solution and FBS (Sigma, USA).

The resulting mixed solution was put into a 50-ml tube (Nunc, USA) containing the scaffold seeded with chondrocytes at the above (i). After being pipetted up and down several times, the content in the tube was loaded into a Teflon mold with the morphology of a biological tissue of interest.

Thereafter, a calcium chloride solution as a cross-linking agent was mixed with HEPES (n-2-Hydroxyethyl)piperazine-N′-[2-ethanesufonic acid]), Sigma, USA) of pH 7.4, and slowly added to the Teflon mold using a 21-gauge syringe for gelation of the scaffold.

For complete gelation of the scaffold, the resulting gel beads were submerged in a calcium chloride solution for 5 min, washed with PBS (Sigma, USA), and transferred to 6-well plates filled with DMEM (JBI, Korea) containing 4 ml of an antibiotic/antifungal solution and FBS (Sigma, USA).

### EXPERIMENTAL EXAMPLES

Evaluation of Cell Proliferation

The gel beads prepared in Example 3 were incubated at 37° C. in high humidity containing 5% CO₂ with 90 rpm of shaker's agitation. The culture medium was replaced by a fresh one every three or four days. On Days 7, 14, 21 and 31, to evaluate chondrocyte proliferation, the gel beads were observed under a scanning electron microscope (S-800, Hitachi, Japan), and while a quantitative DNA assay was measured by fluorescence using a luminescence spectrometer (Luminescence Spectrometer LS50B, PERKIN ELMER, Great British).

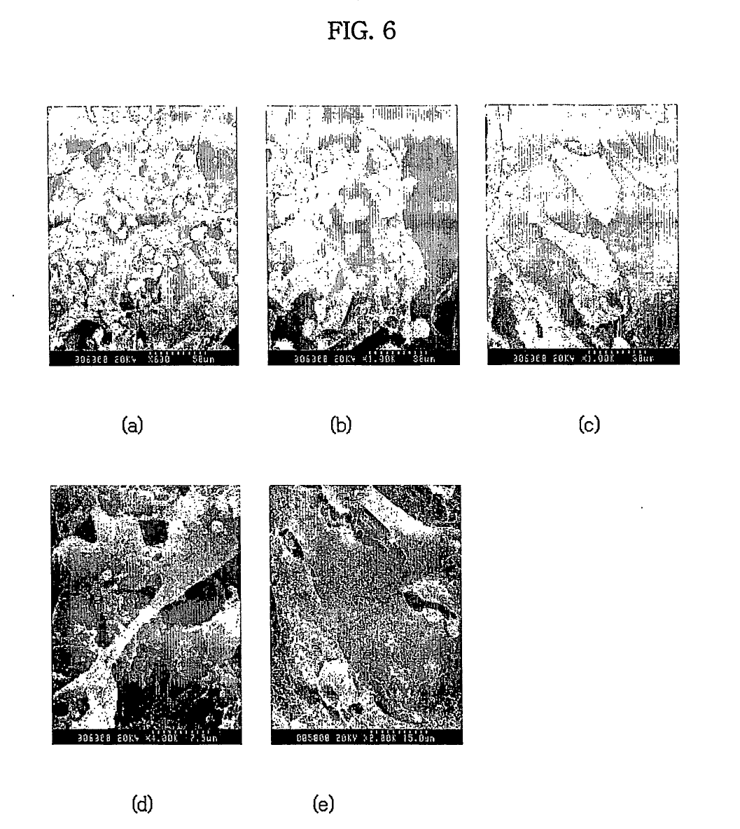

The results are given in FIGS. 6, 7 and 8.

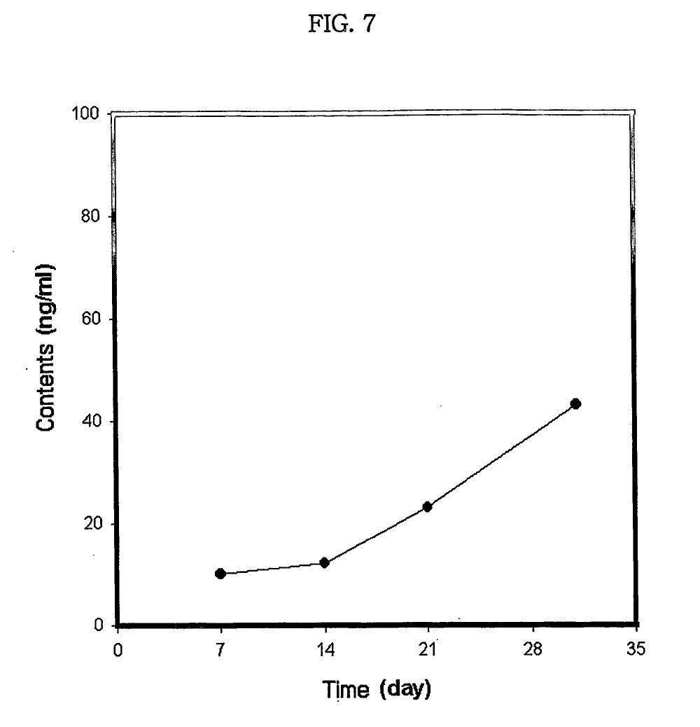

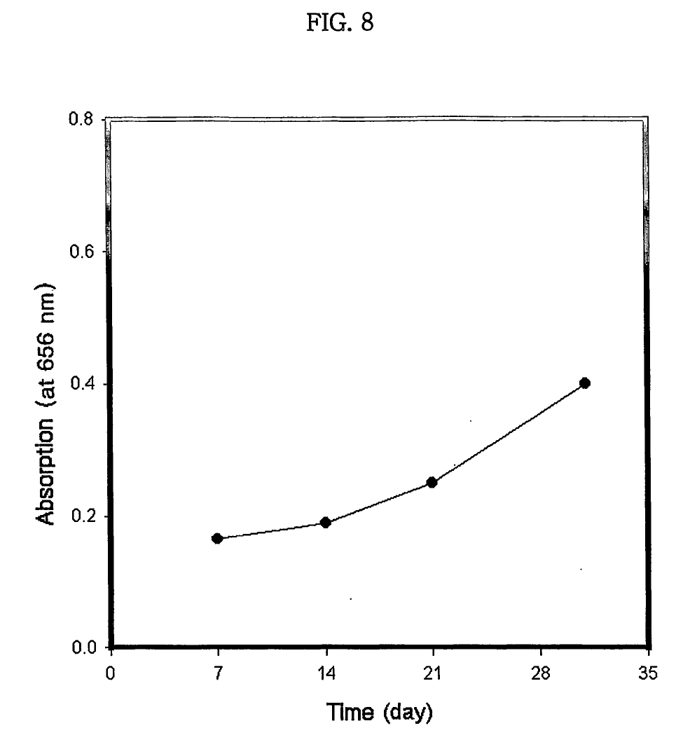

FIG. 6 shows SEM images showing chondrocytes that have been proliferated on the scaffold having a semi-permeable membrane on an outer surface thereof according to the present invention. FIG. 7 is a graph showing DNA synthesis of chondrocytes grown on the scaffold according to the present invention. FIG. 8 is a graph showing glycosaminoglycan synthesis of chondrocytes grown on the scaffold according to the present invention.

In FIG. 6, (a) shows a surface of the scaffold incubated for 7 days; (b) shows an inner surface with porous interconnections of the scaffold incubated for 7 days; (c) shows a surface of the scaffold incubated for 14 days; (d) shows a surface of the scaffold incubated for 21 days; and (e) shows a surface of the scaffold incubated for 31 days.

The secretion of glycosaminoglycan by chondrocytes, as shown in FIG. 8, indicates that chondrocytes normally proliferates while displaying their original function in cartilage tissue.

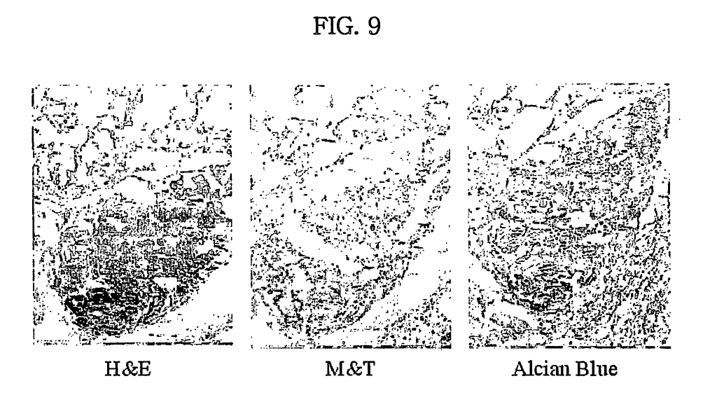

These results reveal that indeed chondrocytes attach and proliferate on both the outer surface and the inner surface of and the scaffold, with cartilage-forming activity. Evaluation of the scaffold for transplantation into animals The scaffold seeded with chondrocytes having a semi-permeable membrane on an outer surface thereof, prepared in the above (ii) of Example 2, was transplanted under the epidermis of the back of nude mice. Chondrocyte proliferation was examined, and the results are given in FIG. 9.

FIG. 9 shows results of hematoxylin-eosin staining, masson-trichrome staining and Alcian Blue staining of the transplanted scaffolds, which were retrieved from the nude mice four weeks after transplantation into the nude mice. In FIG. 9, H&E shows the result of the hematoxylin-eosin staining; M&E shows the result of the masson-trichrome staining; and Alcian Blue shows the result of the Alcian Blue staining.

As shown in FIG. 9, the chondrocytes seeded onto the scaffold having a semi-permeable membrane on an outer surface thereof were found to normally proliferate in vivo.

The scaffold covered with a semi-permeable membrane according to the present invention selectively introduces nutrients into the scaffold by allowing penetration of only external nutrients into the scaffold and excreting metabolic wastes generated by tissue cells to the outside of the scaffold. In addition, the scaffold has the morphology of a biological tissue of interest through cross-linking the small-sized scaffolds, thereby allowing uniform proliferation of tissue cells throughout the whole scaffold.

1. A scaffold for regenerating a biological tissue by seeding tissue cells onto the scaffold and growing the tissue cells on the scaffold, comprising a semi-permeable membrane formed on an outer surface thereof and is 1 to 3 mm in size.

2. The scaffold as set forth in claim 1, wherein the semi-permeable membrane is made of one selected from among alginates, polysaccharides, chitosan, agar powder and gelatin.

3. (Deleted)

4. A method for preparing a scaffold comprising a semi-permeable membrane, comprising:
loading one or more scaffolds into a mold with a predetermined form and size; and
adding a mixture of a semi-permeable agent and a cross-linking agent to the mold and cross-linking the semi-permeable agent to form the semi-permeable membrane on an outer surface of each of the scaffolds.

5. The method as set forth in claim 4, wherein the semi-permeable agent is selected from among alginates, polysaccharides, chitosan, agar powder and gelatin.

6. The method as set forth in claim 4, wherein the cross-linking agent is selected from among calcium chloride, tripolyphosphate and glutaraldehyde.

7. The method as set forth in claim 4, wherein the mold is made of Teflon.

8. A method of preparing a biological tissue, comprising:
seeding cells obtained from a tissue to be regenerated onto one or more scaffolds;
loading the scaffolds seeded with the tissue cells into a molding container with a predetermined form and size;
adding a semi-permeable agent and cross-linking agent to the molding container and forming a semi-permeable membrane on an outer surface of each of the scaffolds loaded in the molding container to interconnect the scaffolds; and
introducing nutrients into the scaffolds interconnected with the cross-linking agent, thus proliferating the tissue cells.

9. The method as set forth in claim 8, wherein the semi-permeable agent is selected from among alginates, polysaccharides, chitosan, agar powder and gelatin.

10. The method as set forth in claim 8, wherein the cross-linking agent is selected from among calcium chloride, tripolyphosphate and glutaraldehyde.

11. The method as set forth in claim 8, wherein the mold is made of Teflon.

12. A biological tissue prepared using the scaffold comprising the semi-permeable membrane according to of claim 1.

13. A biological tissue prepared by the method according to claim 8.
